(19) United States
(12) Patent Application Publication
(10) Pub. No.: US 2022/0084647 A1
(43) Pub. Date: Mar. 17, 2022

(54) MONITORING-BASED USAGE SUGGESTION SYSTEM

(71) Applicant: Radient Technologies Innovations Inc., Edmonton (CA)

(72) Inventors: Michael Cabigon, Edmonton (CA); Jim Seethram, Edmonton (CA); Steven Splinter, Edmonton (CA); Denis Taschuk, Edmonton (CA)

(21) Appl. No.: 17/240,557

(22) Filed: Apr. 26, 2021

Related U.S. Application Data
(63) Continuation of application No. PCT/IB2019/058792, filed on Oct. 14, 2019.
Application No. PCT/IB2019/058792 is a continuation of application No. 62/750,210, filed on Oct. 24, 2018.

(51) Int. Cl. G16H 20/10; G16H 50/30; A61B 5/16; A61B 5/00

(52) U.S. Cl. CPC G16H 20/10; A61B 5/4839; A61B 5/165; G16H 50/30

(57) ABSTRACT

Systems and methods are provided for identifying the best strain of cannabis to consume in order to combat different types of stress. The system includes a recommendation network that makes a recommendation to the user based on the user's stress. The system further includes a user device, an intake device, and one or more wearable stress sensors.

### CROSS-REFERENCE TO RELATED APPLICATIONS

The present patent application is a continuation of International Application No. PCT/IB2019/058792 filed Oct. 15, 2019, which claims the priority benefit of U.S. provisional patent No. 62/750,210 filed Oct. 24, 2018, the disclosures of which are incorporated by reference herein.

### BACKGROUND OF THE INVENTION

### 1. Field of Disclosure

The present disclosure is generally related to providing cannabis recommendations to users based on physiological values in real time.

### 2. Description of the Related Art

Cannabis contains a unique class of terpeno-phenolic compounds known as cannabinoids or phytocannabinoids. The principle cannabinoids present in cannabis are tetrahydrocannabinol (THC), which is a potent psychoactive cannabinoid and cannabidiol (CBD), which is non-psychoactive but is widely known to have therapeutic potential for a variety of medical conditions. The proportion of cannabinoids in the plant may vary from strain to strain. Based on the proportion of the cannabinoids present in a plant variety, the psychoactive and medicinal effects obtained from different plant varieties may vary. Such variance is further exacerbated by the presence of certain terpenoid or phenolic compounds which may be present in the plant, which may also have pharmacological activity.

Cannabis can reduce stress and anxiety in users. However, the variety of cannabis strains is as wide as the variety of stressors in the world. Given how effects of cannabis can vary greatly from strain to strain, it is important for users to identify the best strain for a given situation.

### SUMMARY OF THE CLAIMED INVENTION

The present disclosure is a wearable sensor technology that quantifies and categorizes stress to recommend the correct strain of cannabis to a user in a given situation. The present disclosure is a method of providing cannabis users with more effective stress relief by using a recommendation network to collect cannabis usage and stress sensor data and correlate stress reduction to specific cannabis products.

### BRIEF DESCRIPTIONS OF THE DRAWINGS

FIG. 1 illustrates a system for suggesting cannabis usage based on monitored stress levels, according to various embodiments.

FIG. 2 illustrates a monitoring module, according to various embodiments.

FIG. 3 illustrates a correlation module, according to various embodiments.

FIG. 4 illustrates a recommendation module, according to various embodiments.

### DETAILED DESCRIPTION

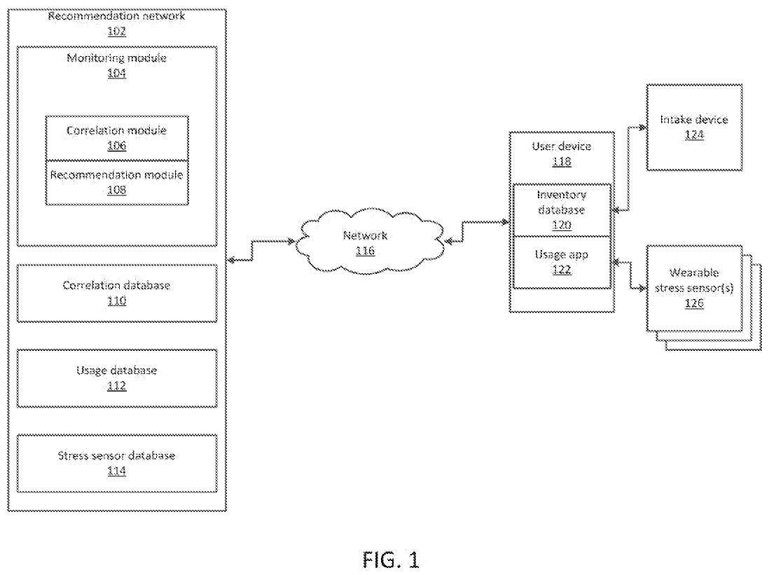

FIG. 1 illustrates a system for suggesting cannabis usage based on monitored stress levels, according to various embodiments. The system includes a recommendation network 102 that collects cannabis usage and stress sensor data from a plurality of users who have an intake device (e.g., vaporizer) 124 that can communicate cannabis consumption data to the recommendation network 102. The system includes at least one wearable device that can quantify and classify stress experienced by the user, and correlates stress reduction to specific cannabis products in order to make a recommendation specific to the user's current amount and type of stress. A monitoring module 104 is part of the recommendation network 102 and is constantly polling a stress sensor database 114 for new data events. When an event is detected, a correlation module 106 is called to update the correlation database 110. When stress is present, the correlation module 106 calls the recommendation module 108 to recommend the cannabis strain that is in the user's inventory that is most highly correlated with a reduction in the type of stress being currently detected. The correlation module 106 is called by the monitoring module 104 when a new data event appears in the stress sensor database 114. The correlation module 106 updates the correlation coefficients between stress reduction and cannabis strain(s) consumed. A recommendation module 108 is called by the monitoring module 104 when the stress sensor data indicates the user is stressed. The recommendation module 108 utilizes a correlation database 110 to recommend the strain in the inventory database 120 that has the highest correlation coefficient with a reduction in the current type of measured stress. The correlation database 110 is populated by the correlation module 106 with the correlations between cannabis strain consumption and a reduction in a type of stress (e.g., acute or chronic). A usage database 112 is populated by the intake device, cataloging each user's cannabis consumption history, including strain and quantity. A stress sensor database 114 is populated by a wearable stress sensor 126, cataloging the users' stress level over time.

A cloud or communication network 116 may be a wired and/or a wireless network. The network 116, if wireless, may be implemented using communication techniques such as visible light communication (VLC), worldwide interoperability for microwave access (WiMAX), long term evolution (LTE), wireless local area network (WLAN), infrared (IR) communication, public switched telephone network (PSTN), radio waves, and other communication techniques known in the art. The network 116 may allow ubiquitous access to shared pools of configurable system resources and higher-level services that can be rapidly provisioned with minimal management effort, often over Internet and relies on sharing of resources to achieve coherence and economies of scale, like a public utility, while third-party clouds enable organizations to focus on their core businesses instead of expending resources on computer infrastructure and maintenance.

A user device 118 (e.g., laptop, smartphone, tablet, smart watch, etc.) facilitates the collection of data from the intake device 124 and the wearable stress sensor(s) 126 and transmits that data via its communication hardware to the recommendation network 102. An inventory database 120 is populated by the user, cataloging the amount of and type of cannabis products they currently have. In other embodiments, this would be populated by point of sale systems at cannabis stores or dispensaries. A usage app 122, such as Releaf, allows the user to track their cannabis usage and populate the inventory database 120. Other usage applications may also be used to receive data from the user, as well as to communicate data (e.g., a recommendation) to the user by way of a displayed message or notification.

In one example, the intake device 124 is a vaporizing device for delivering cannabis products to the user, while monitoring use levels and communicating that information wirelessly to another device. One or more wearable stress sensor(s) 126 can quantify the level of stress a wearer is experiencing (e.g., on a scale of 1-10) and categorize the stress into at least two different types (e.g., chronic or acute).

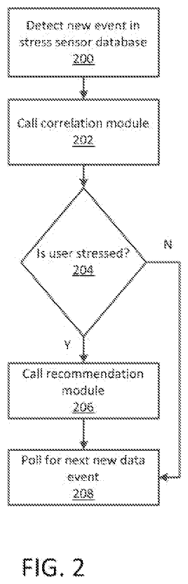

FIG. 2 illustrates a monitoring module, according to various embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The process begins at 200 with a new data event detected in the stress sensor database 114. At step 202, the monitoring module 104 calls the correlation module 106 to update the correlation coefficients between consumption of a specific cannabis strain (e.g., strain A, B or C) and a reduction in a specific type of stress (e.g., 6 out of 10 and acute). At 204, the monitoring module 104 determines if the user is stressed from the current wearable stress sensor data in the stress sensor database 114. If the user is determined to be stressed, the recommendation module 108 is called at 206. At 208, the monitoring module 104 then polls for the next new data event in the stress sensor database 114 to begin the process again.

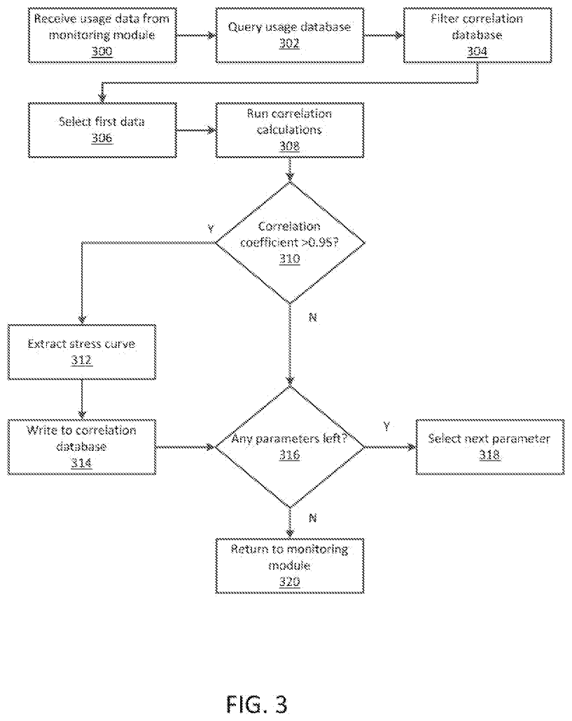

FIG. 3 illustrates a correlation module, according to various embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The process begins at 300 when usage data is received from the monitoring module 104. At 302, the usage database 112 is queried for the cannabis strain the user is currently using. At 304, the correlation database 110 is filtered for correlations related to the current strain. At 306, the first data (e.g., level 6 out of 10 acute stress) is selected. At 308, the correlation calculations are run for all the data that has the same level and type of stress. At 310, it is determined if there is a correlation coefficient greater than 0.95 (an arbitrarily chosen threshold for the purposes of this example). If the correlation coefficient is above the predetermined threshold, the steepest downward sloping stress curve is selected and extracted (e.g., strain A 50 mg) at 312. At 314, the identified data point is written to correlation database 110. At 316, it is determined if there are any parameters left. If yes, the next parameter is examined at 318. If no, the process returns to the monitoring module 104 at 320.

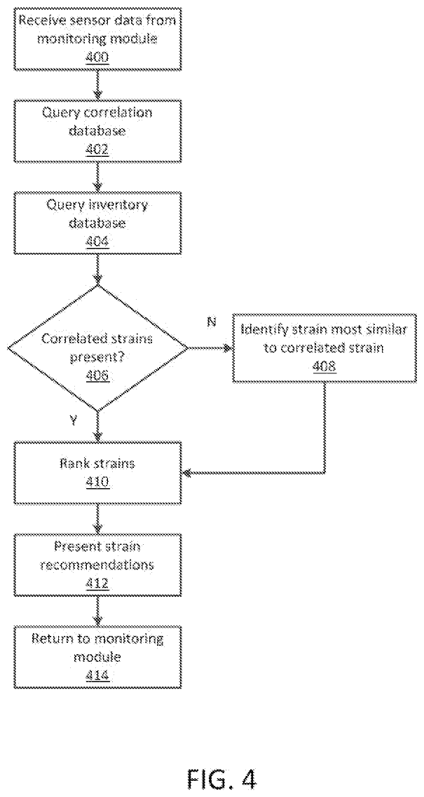

FIG. 4 illustrates a recommendation module, according to various embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The process begins at 400 when stress data is received from the monitoring module 104. At 402, the correlation database 110 is queried for the cannabis strain(s) correlated to a downward sloping curve of stress data produced by the user's wearable stress sensor(s) 126. At 404, the inventory database 120 is queried for any of the cannabis strains identified as correlated to downward sloping stress curves. At 406, it is determined if any correlated strains are present in the inventory database 120. At 408, the cannabis strain in the inventory database 120 that is most similar in attributes (e.g., indica vs. sativa, THC percentage, THC/CBD ratio, etc.) to correlated strains is identified. At 410, the cannabis strains in the inventory database 120 are ranked by the downward slope of the stress curve in the correlation database 110. At 412, the strain recommendations are presented to the user on the usage app 122. At 414, the process returns to the monitoring module 104.

Although the present disclosure and its advantages have been described in detail, it should be understood that various changes, substitutions and alterations can be made herein without departing from the disclosure as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A method of providing cannabis consumption recommendations, the method comprising:
storing information in an inventory database regarding one or more cannabis products available to a user;
detecting one or more indicators of stress via one or more wearable sensors associated with the user;
generating a recommendation regarding one of the cannabis products available to the user, wherein generating the recommendation is based on the detected stress indicators; and
providing the recommendation to the user.

2. The method of claim 1, further comprising populating the inventory database via a usage application.

3. The method of claim 1, wherein the wearable sensors quantify the detected stress indicators, and further comprising identifying a stress level based on the quantified stress indicators.

4. The method of claim 1, wherein providing the recommendation to the user includes displaying a message that includes the recommendation on a display screen of a user device.

5. The method of claim 1, further comprising providing the recommendation to an intake device that controls consumption of the recommended cannabis product, wherein the intake device controls consumption of the recommended cannabis product in accordance with the recommendation.

6. The method of claim 5, further comprising monitoring the consumption of the recommended cannabis product by the user via the intake device, and sending data regarding the monitored consumption to a recommendation network server.

7. The method of claim 1, further comprising polling the wearable sensors for the detected stress indicators, and storing the detected stress indicators in a correlation database.

8. The method of claim 7, wherein the stress indicators are detected before and after consumption, and further comprising identifying a correlation between the recommended cannabis product and a reduction in stress level based on the detected stress indicators before and after consumption.

9. The method of claim 8, further comprising identifying the reduction in stress level based on a downward pattern in the detected stress indicators over a time following consumption.

10. The method of claim 8, wherein identifying the correlation further comprises:
calculating a correlation coefficient for the one or more available cannabis products, each cannabis product associated with a change in level and type of stress;
filtering correlation data regarding the available cannabis products based on the respective correlation coefficient meeting a predetermined threshold; and
identifying the highest correlation coefficient among the filtered correlation data, wherein the highest correlation coefficient is associated with the recommended cannabis product.

11. The method of claim 10, wherein generating the recommendation comprises:
querying the inventory database regarding the one or more cannabis products available to the user; and
matching the correlated type of stress to the detected stress indicators.

12. The method of claim 1, further comprising ranking the available cannabis products based on the correlation coefficients; and presenting a list of the ranked cannabis products on a display screen of a user device.

13. A system of providing cannabis consumption recommendations, the system comprising:
an inventory database in memory that stores information regarding one or more cannabis products available to a user;
one or more wearable sensors associated with the user, wherein the searable sensors detect one or more indicators of stress;
a recommendation module executable to generate a recommendation regarding one of the cannabis products available to the user, wherein generating the recommendation is based on the detected stress indicators; and
a user device that provides the recommendation to the user.

14. The system of claim 13, wherein the wearable sensors quantify the detected stress indicators, and further comprising a monitoring module executable to identify a stress level based on the quantified stress indicators.

15. The system of claim 13, further comprising an intake device, wherein the recommendation module provides the recommendation to the intake device, and wherein the intake device controls consumption of the recommended cannabis product in accordance with the recommendation.

16. The system of claim 16, wherein the intake device further monitors the consumption of the recommended cannabis product by the user, and sends data regarding the monitored consumption to a recommendation network server.

17. The system of claim 13, further comprising a monitoring module executable to poll the wearable sensors for the detected stress indicators, and a correlation database that stores the detected stress indicators.

18. The system of claim 17, wherein the stress indicators are detected before and after consumption, and wherein the correlation module is further executable to identify a correlation between the recommended cannabis product and a reduction in stress level based on the detected stress indicators before and after consumption.

19. The system of claim 13, wherein the recommendation module is further executable to rank the available cannabis products based on the correlation coefficients; and wherein the user device present a list of the ranked cannabis products on a display screen.

20. A non-transitory, computer-readable storage medium, having embodied thereon a program executable by a processor to perform a method of providing cannabis consumption recommendations, the method comprising:
storing information in an inventory database regarding one or more cannabis products available to a user;
detecting one or more indicators of stress via one or more wearable sensors associated with the user;
generating a recommendation regarding one of the cannabis products available to the user, wherein generating the recommendation is based on the detected stress indicators; and
providing the recommendation to the user.
